(19) United States
(12) Patent Application Publication
(10) Pub. No.: US 2026/0115711 A1
(43) Pub. Date: Apr. 30, 2026

(54) SYSTEM AND METHOD FOR PROVIDING HEALTH CARE SERVICE RELATED TO URINE ANALYSIS

(71) Applicant: DNC BIOTECHNOLOGY INC., Daejeon (KR)

(72) Inventors: Seong Su PARK, Anseong-si (KR); Hyun Kyung SONG, Seoul (KR)

(21) Appl. No.: 19/375,288

(22) Filed: Oct. 31, 2025

(30) Foreign Application Priority Data
Oct. 31, 2024 (KR) 10-2024-0152140

(51) Int. Cl. B01L 3/00; A61B 5/20

(52) U.S. Cl. CPC B01L 3/5023; B01L 3/508; A61B 5/207; B01L 2300/027; B01L 2300/0654; B01L 2300/069

(57) ABSTRACT

A system for providing a health care service related to urine analysis including a urine analysis device; and an electronic device is provided. The electronic device is configured to: transmit a start signal for the urine analysis to the urine analysis device; present a screen related to a progress status of the urine analysis based on a signal related to the progress status of the urine analysis and received from the urine analysis device; and present a result screen of the urine analysis based on a signal related to a result of the urine analysis and received from the urine analysis device. The urine analysis device is configured to: perform urine analysis based on color information of test papers in response to the start signal for the urine analysis; transmit the signal related to the progress status of the urine analysis to the electronic device; and transmit the signal related to the result of the urine analysis to the electronic device.

### CROSS-REFERENCE TO RELATED APPLICATION

This application claims the benefit under 35 USC 119 (a) of Korean Patent Applications No. 10-2024-0152140 filed on Oct. 31, 2024 in the Korean Intellectual Property Office, the entire disclosures of which are incorporated herein by reference for all purposes.

### TECHNICAL FIELD

The present disclosure relates to a system and method for providing a health care service related to urine analysis.

### BACKGROUND

In general, a urine test is an essential examination that occupies about 30% of the total number of examinations performed in most hospitals or clinics. The urine test is a method to examine the components of urine to assess systemic conditions and detect lesions.

A conventional urine test is performed to examine urine by bringing a test stick with a paper test strip into contact with an examinee's urine and checking a color change of the paper test strip. That is, an examiner performs the urine test by directly applying the examinee's urine to the test stick, or by collecting urine in a container, such as a paper cup, and bringing the test stick into contact with the collected urine.

When a urine test is performed by using a urine analysis device including a urine test kit equipped with a flat test strip, the test is performed by emitting light over the entire area of the flat test strip and receiving the reflected light to determine the color. However, when the test is performed by using a urine test kit equipped with a curved test strip, it is difficult to determine the color of the entire area of the curved test strip by the conventional test method.

Also, if urine test kits are manufactured with varying degrees of curvature, such as differences in the radius of curvature or radius, urine analysis devices capable of accurate detection are required for each type.

Further, there is a demand for a device that displays to a user a result of urine analysis using a urine analysis device, or that provides the user with a health care service related to the urine analysis.

### PRIOR ART DOCUMENT

### Patent Document

(Patent Document 1) Korean Patent No. 10-1095280 (registered on Dec. 11, 2011)

### SUMMARY

In view of the foregoing, the present disclosure is conceived to provide an electronic device configured to display a result screen according to a result of urine analysis received from a urine analysis device.

Also, the present disclosure is conceived to provide an electronic device configured to display a urine analysis result screen including urine analysis items categorized based on severity levels.

Further, the present disclosure is conceived to provide an electronic device configured to provide a comprehensive health care service including not only urine analysis but also a user's urination log and water intake information.

However, the problems to be solved by the present disclosure are not limited to the above-described problems. There may be other problems to be solved by the present disclosure.

An aspect of the present disclosure provides a system for providing a health care service related to urine analysis, including: a urine analysis device that includes a curved test strip which is attached along an inner or outer circumferential surface of a container into which urine is collected and includes a plurality of test papers provided to test the collected urine, a sensor unit which includes a light emitting unit configured to emit light onto the plurality of test papers and a light receiving unit configured to detect a color of each test paper based on the light reflected from each of the plurality of test papers, and an analysis unit which analyzes the urine based on the color information of the test papers detected by the light receiving unit; and an electronic device that includes a communication circuit configured to directly or indirectly transmit and receive signals with the urine analysis device, a display, and a touch panel integrally coupled with the display. The electronic device is configured to: transmit, through the communication circuit, a start signal for the urine analysis to the urine analysis device; present, on the display, a screen related to a progress status of the urine analysis based on a signal related to the progress status of the urine analysis and received from the urine analysis device; and present, on the display, a result screen of the urine analysis based on a signal related to a result of the urine analysis and received from the urine analysis device. The urine analysis device is configured to: perform urine analysis based on the color information of the test papers in response to the start signal for the urine analysis; transmit the signal related to the progress status of the urine analysis to the electronic device; and transmit the signal related to the result of the urine analysis to the electronic device.

Another aspect of the present disclosure provides a method for providing a health care service related to urine analysis, including: transmitting a start signal for the urine analysis to a urine analysis device by an electronic device; performing urine analysis in response to the start signal by the urine analysis device; transmitting a signal related to a progress status of the urine analysis to the electronic device by the urine analysis device; presenting a screen related to the progress status of the urine analysis based on the signal related to the progress status and received from the urine analysis device by the electronic device; transmitting a signal related to a result of the urine analysis to the electronic device by the urine analysis device; and presenting a result screen of the urine analysis based on the signal related to the result of the urine analysis and received from the urine analysis device by the electronic device. The urine analysis device includes a curved test strip which is attached along an inner or outer circumferential surface of a container into which urine is collected and includes a plurality of test papers provided to test the collected urine, a sensor unit which includes a light emitting unit configured to emit light onto the plurality of test papers and a light receiving unit configured to detect a color of each test paper based on the light reflected from each of the plurality of test papers, and an analysis unit which analyzes the urine based on the color information of the test papers detected by the light receiving unit.

According to the present disclosure, it is possible to display, to a user, a result screen according to a result of urine analysis received from a urine analysis device, and to display urine analysis items categorized based on severity levels.

Also, according to the present disclosure, it is possible to provide a comprehensive health care service including not only urine analysis but also a user's urination log and water intake information.

### BRIEF DESCRIPTION OF THE DRAWINGS

In the detailed description that follows, embodiments are described as illustrations only since various changes and modifications will become apparent to a person with ordinary skill in the art from the following detailed description. The use of the same reference numbers in different drawings indicates similar or identical items.

FIG. 1 is a perspective view of a urine analysis device according to an embodiment of the present disclosure.

FIG. 2 shows the urine analysis device with its outer cover opened according to an embodiment of the present disclosure.

FIG. 3 is an exploded perspective view of the urine analysis device according to an embodiment of the present disclosure.

FIG. 4 shows an outer case of the urine analysis device according to an embodiment of the present disclosure.

FIG. 5 shows an inner case of the urine analysis device according to an embodiment of the present disclosure.

FIG. 6A shows a container of the urine analysis device according to an embodiment of the present disclosure.

FIG. 6B shows a curved test strip attached to the container of the urine analysis device according to an embodiment of the present disclosure.

FIG. 7A and FIG. 7B are diagrams for explaining a method of acquiring color information of each of a plurality of test papers by emitting light from the urine analysis device according to an embodiment of the present disclosure.

FIG. 8 is a configuration view of a system for providing a health care service related to urine analysis according to an embodiment of the present disclosure.

FIG. 9 is a configuration view of an electronic device for providing a health care service related to urine analysis according to an embodiment of the present disclosure.

FIG. 10A shows a login screen displayed on the electronic device according to an embodiment of the present disclosure.

FIG. 10B shows a main screen displayed on the electronic device according to an embodiment of the present disclosure.

FIG. 11A shows a test start screen displayed on the electronic device according to an embodiment of the present disclosure.

FIG. 11B shows a screen displayed on the electronic device during testing according to an embodiment of the present disclosure.

FIG. 11C shows a result confirmation screen displayed on the electronic device according to an embodiment of the present disclosure.

FIG. 12A to FIG. 12C are screens showing urine analysis results displayed on the electronic device according to an embodiment of the present disclosure.

FIG. 13A to FIG. 13C are screens related to a urination log displayed on the electronic device according to an embodiment of the present disclosure.

FIG. 14A to FIG. 14C are screens related to water intake displayed on the electronic device according to an embodiment of the present disclosure.

FIG. 15 is a screen for providing medical-institution information displayed on the electronic device according to an embodiment of the present disclosure.

FIG. 16 is a screen for providing telemedicine displayed on the electronic device according to an embodiment of the present disclosure.

FIG. 17 is a flowchart showing a method for providing a health care service related to urine analysis according to an embodiment of the present disclosure.

### DETAILED DESCRIPTION

Hereinafter, embodiments will be described in detail with reference to the accompanying drawings so that the present disclosure may be readily implemented by a person with ordinary skill in the art. However, it is to be noted that the present disclosure is not limited to the embodiments but can be embodied in various other ways. In the drawings, parts irrelevant to the description are omitted for the simplicity of explanation, and like reference numerals denote like parts throughout the whole document.

Throughout the whole document, the term “on” that is used to designate a position of one element with respect to another element includes both a case that the one element is adjacent to the other element and a case that any other element exists between these two elements.

Throughout the whole document, it is to be understood that the term “comprises or includes” and/or “comprising or including” used in the document means that one or more other components, steps, operation and/or existence or addition of elements are not excluded in addition to the described components, steps, operation and/or elements unless context dictates otherwise and is not intended to preclude the possibility that one or more other features, numbers, steps, operations, components, parts, or combinations thereof may exist or may be added.

Throughout the whole document, the term “about or approximately” or “substantially” is intended to have meanings close to numerical values or ranges specified with an allowable error and intended to prevent accurate or absolute numerical values disclosed for understanding of the present disclosure from being illegally or unfairly used by any unconscionable third party. Throughout the whole document, the term “step of” does not mean “step for.”

Throughout the whole document, the term “combination(s) of” included in Markush type description means mixture or combination of one or more components, steps, operations and/or elements selected from a group consisting of components, steps, operation and/or elements described in Markush type and thereby means that the disclosure includes one or more components, steps, operations and/or elements selected from the Markush group.

Throughout the whole document, a phrase in the form “A and/or B” means “A or B, or A and B.”

Hereinafter, embodiments and examples of the present disclosure will be described in detail with reference to the accompanying drawings. However, the present disclosure may not be limited to the following embodiments, examples, and drawings.

Hereinafter, embodiments of the present disclosure will be described in detail with reference to the accompanying drawings.

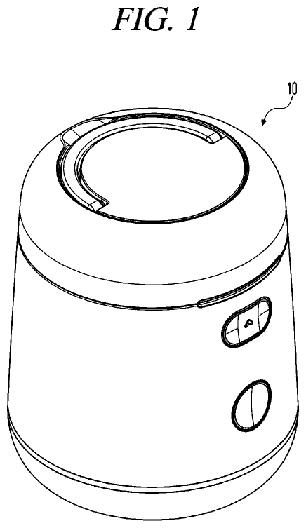

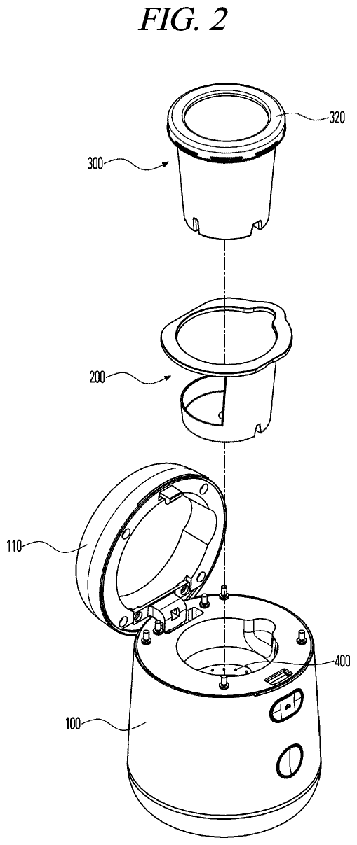

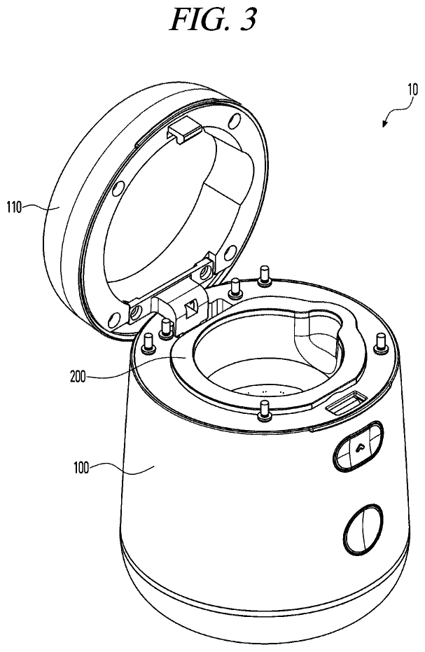

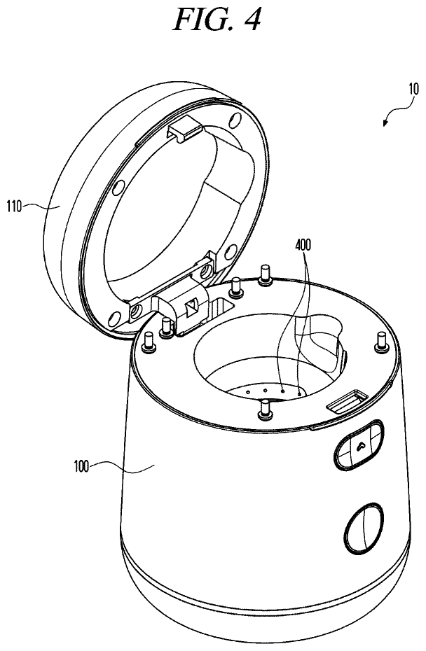

FIG. 1 is a perspective view of a urine analysis device 10 according to an embodiment of the present disclosure, FIG. 2 shows the urine analysis device 10 with its outer cover 110 opened according to an embodiment of the present disclosure, FIG. 3 is an exploded perspective view of the urine analysis device 10 according to an embodiment of the present disclosure, and FIG. 4 shows an outer case 100 of the urine analysis device 10 according to an embodiment of the present disclosure.

Referring to FIG. 1 to FIG. 4, the urine analysis device 10 according to an embodiment of the present disclosure is configured to analyze urine collected into a container 300 from an examinee by detecting the color of a test paper 311 of a curved test strip 310 which reacts with the urine of the examinee.

The urine analysis device 10 may include the outer case 100, an inner case 200, the container 300, a sensor unit 400, an analysis unit (not shown), a measurement unit (not shown), and a correction unit (not shown), but is not limited thereto. For example, the urine analysis device 10 is merely an embodiment of the present disclosure, and various modifications are possible. Herein, the measurement unit (not shown) and the correction unit (not shown) may be implemented by an MCU with built-in memory and peripherals. The built-in memory may store firmware and a standard colorimetric table containing reference color values for each test item.

As shown in FIG. 1 to FIG. 4, the outer case 100 is open at the top such that the inner case 200 can be inserted from above, and may be formed into a cylindrical shape.

A power button configured to turn the power ON and OFF may be provided on an outer surface of the outer case 100, and the sensor unit 400 may be formed on a part of an inner wall of the outer case 100.

The urine analysis device 10 may include the outer cover 110 coupled to the outer case 100 and configured to seal the outer case 100 and the inner case 200.

For example, as shown in FIG. 2 to FIG. 4, the outer cover 110 may be hinge-coupled to one side of an upper portion of the outer case 100, and coupled to the outer case 100 through a fixing portion formed on the other side of the upper portion of the outer case 100. However, the present disclosure is not limited thereto. The outer cover 110 and the outer case 100 may be rotatably coupled to each other.

Further, since a handle is rotatably coupled to a groove formed into a U-shape on an upper portion of the outer cover 110, a user can conveniently carry the urine analysis device 10.

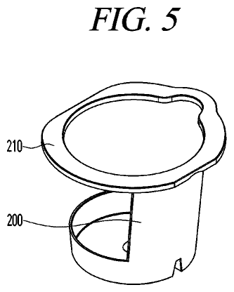

FIG. 5 shows the inner case 200 of the urine analysis device 10 according to an embodiment of the present disclosure.

As shown in FIG. 2 and FIG. 5, the inner case 200 may be detachably disposed inside the outer case 100.

The inner case 200 may include a retaining part 210 protruding outward along an upper outer periphery of the inner case 200, and the retaining part 210 may be mounted on an upper end portion of the outer case 100.

Thus, since the retaining part 210 protruding outward at an upper end portion of the inner case 200 is mounted on the upper end portion of the outer case 100, the user may grip the retaining part 210 to easily attach or detach the inner case 200 mounted inside the outer case 100.

Meanwhile, a portion of a side surface of the inner case 200 corresponding to the sensor unit 400 may be openable.

For example, as shown in FIG. 2, FIG. 3, and FIG. 5, since the portion of the side surface of the inner case 200 corresponding to the sensor unit 400 is open, the sensor unit 400 formed on the part of the inner wall of the outer case 100 may emit light onto the test paper 311 of the test strip provided in the container 300.

In this case, a lower end portion of the opened side surface of the inner case 200 may remain closed by a predetermined height from a lower end portion of the inner case 200 such that the container 300 mounted in the inner case 200 can be fixed.

Meanwhile, the outer case 100 may be formed to have a height sufficient to cover the test paper 311 attached to the container 300. For example, the outer case 100 may have a substantially cylindrical accommodating space capable of accommodating the entire inner case 200 and container 300. For example, the accommodating space may have a cylindrical shape concentric with the container 300, and the portion of the side surface of the inner case 200 corresponding to the sensor unit 400 may be open, which facilitates alignment between the sensor unit 400 and the curved test strip 310.

After the inner case 200 and the container 300 are accommodated in the accommodating space of the outer case 100, the accommodating space can be sealed with the outer cover 110 to suppress the introduction of external light into the accommodating space.

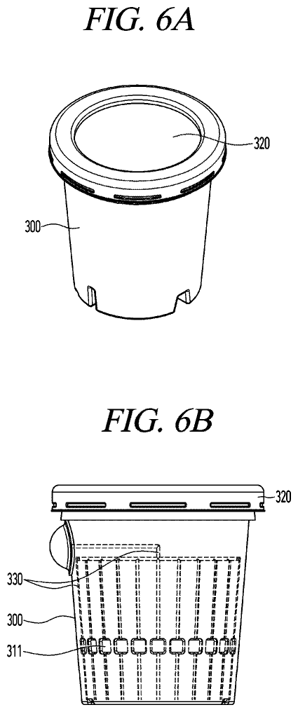

FIG. 6A shows the container 300 of the urine analysis device 10 according to an embodiment of the present disclosure, and FIG. 6B shows the curved test strip 310 attached to the container 300 of the urine analysis device 10 according to an embodiment of the present disclosure.

Referring to FIG. 6A and FIG. 6B, the container 300 collects urine and may be mounted in the inner case 200. The container 300 may include the curved test strip 310 provided along an inner or outer circumferential surface of the container 300, and the curved test strip 310 may include a plurality of test papers 311 for testing the urine collected in the container 300. Although FIG. 6A and FIG. 6B show the curved test strip 310 attached along the inner circumferential surface of the container 300, the present disclosure is not limited thereto. The curved test strip 310 may be attached along the outer circumferential surface.

For example, the curved test strip 310 may be attached along the inner or outer circumferential surface of the container 300 with an appropriate curvature, depending on the size of the container 300 and the length of the curved test strip 310.

The curved test strip 310 may individually accommodate test papers 311, which react with urine, in separate compartments, and may include flow paths 330 that allow urine to flow into the respective compartments.

In this case, a plurality of test papers 311 may be formed corresponding in number to various test items. For example, the test papers 311 may be 11, 7, or 4 in number, as shown in Table 1.

TABLE 1

| | | Initial | 11-Point | 7-Point | 4-Point |
|---|---|---|---|---|---|
| No. | Test Item | Color | Kit | Kit | Kit |
| 1 | Occult Blood | Color a | ∘ | ∘ | ∘ |
| 2 | Bilirubin | Color b | ∘ | — | — |
| 3 | Urobilinogen | Color c | ∘ | ∘ | — |
| 4 | Ketones | Color d | ∘ | ∘ | — |
| 5 | Protein | Color e | ∘ | — | ∘ |
| 6 | Nitrite | Color f | ∘ | ∘ | — |
| 7 | Glucose | Color g | ∘ | ∘ | ∘ |
| 8 | pH | Color h | ∘ | ∘ | — |
| 9 | Specific | Color i | ∘ | — | — |
| | Gravity | | | | |
| 10 | Leukocytes | Color j | ∘ | ∘ | ∘ |
| 11 | Vitamin C | Color k | ∘ | — | — |

Referring to Table 1, the test items may include occult blood (OBD), bilirubin (BIL), urobilinogen (URO), ketones (KET), protein (PRO), nitrite (NIT), glucose (GLU), pH, specific gravity (SG), leukocytes (LEU), and vitamin C. Urine analysis can be performed by using the container 300 which includes 11, 7, or 4 test papers 311 depending on the purpose of the test. The flow path 330 may be divided into a lower flow path connected to a lower side of the test paper 311 and an upper flow path connected to an upper side of the test paper 311. When an air pump (not shown) attached to the upper flow path is pressed, urine collected in the container 300 flows through the lower flow path and wets the test paper 311.

The container 300 may be cylindrical or cup-shaped and formed of a transparent plastic material to facilitate urine collection from the examinee and to allow visual inspection of the collected urine.

A container cover 320 may be coupled to an upper portion of the container 300 to suppress leakage of the urine collected from the examinee to the outside.

When the container 300 is mounted inside the inner case 200, a protrusion or groove (not shown) for guiding the container 300 to a correct mounting position may be formed at the bottom of the container 300. Conversely, a groove or protrusion corresponding to the protrusion or groove (not shown) of the container 300 may be formed in the inner case 200.

Meanwhile, a tag or code for recording information of the examinee may be attached to one surface of the container 300.

For example, an NFC reader (not shown) may be attached to the one surface of the container 300. The NFC reader is configured to read recorded data from an NFC tag attached to the container 300. The NFC tag may record information, such as the examinee's data and the type of container 300 indicating whether it is a 11-point kit, a 7-point kit, or a 4-point kit. Although the NFC tag has been described as an example in the embodiment of the present disclosure, RFID, barcodes, or QR codes may also be used.

The urine analysis device 10 of the present embodiment may be configured to allow the container 300 to be mounted inside the inner case 200. Once the container 300 is mounted, the urine analysis device 10 emits light onto the test papers 311 of the curved test strip 310, receives light reflected from the test papers 311, and analyzes the urine based on color information of the reflected light. The urine analysis device 10 may include a display to present a result of urine analysis.

The urine analysis device 10 according to the present embodiment can accurately perform a urine test even when the urine container 300 equipped with the curved test strip 310 is used.

Further, the urine analysis device 10 according to an embodiment of the present disclosure can derive accurate test results by adjusting a test start time of each test item through individual scanning of a plurality of sensor units 400.

For example, after urine adheres to each test paper, scanning may be performed for glucose and bilirubin after 30 seconds, for ketone after 40 seconds, for specific gravity after 45 seconds, for pH, protein, urobilinogen, blood, and nitrite after 60 seconds, and for leukocytes after 2 minutes.

Furthermore, according to an embodiment of the present disclosure, more accurate test results may be derived through individual scanning of the plurality of sensor units 400, and the number and cycle of scanning for each test item may be varied accordingly.

Meanwhile, the sensor unit 400 may include a light emitting unit 410 configured to emit light onto the plurality of test papers 311 and a light receiving unit 420 configured to detect a color of each test paper 311 based on the light reflected from each of the plurality of test papers 311.

The sensor unit 400 and the curved test strip 310 may be arranged as concentric circles with different radii, assuming that each of the sensor unit 400 and the curved test strip 310 is extended with the same curvature. Accordingly, each test paper 311 of the curved test strip 310 can be aligned to face a corresponding sensor unit 400.

The sensor unit 400 may include a plurality of light emitting units 410 configured to emit light onto each of the plurality of test papers 311, and a plurality of light receiving units 420 configured to detect colors of light reflected from each of the plurality of test papers 311.

A plurality of sensor units 400 may be provided on an inner surface of the outer case 100, and each sensor unit 400 may include a pair of a light emitting unit 410 and a light receiving unit 420.

For example, the sensor units 400 may be arranged on at least a part of the inner wall of the outer case 100 and oriented toward the center of the accommodating space of the outer case 100. Thus, when the inner case 200 and the container 300 are mounted in the outer case 100, the sensor units 400 are aligned to face the curved test strip 310 and can scan the test papers 311.

In an embodiment, the sensor units 400 are formed on a flexible printed circuit board (FPCB) and can be easily located on the inner wall of the outer case 100. That is, the FPCB may be disposed on at least a part of the inner wall in the cylindrical accommodating space of the outer case 100, and the sensor units 400 may be formed on the FPCB disposed on the inner wall in the cylindrical accommodating space.

The light emitting unit 410 may emit light onto the test paper 311 of the test strip provided in the container 300, and may be configured as, for example, a white LED sensor. For example, the light emitting unit 410 may be composed of a high-brightness white LED driven by a constant current control method and a MOSFET capable of changing a current applied to the LED in response to a PWM control signal from the MCU.

In an embodiment, the LED may be a high-brightness white LED with a color temperature of 5600K.

As described above, the sensor units 400 are aligned to face the curved test strip 310, and, thus, the plurality of light emitting units 410 is positioned to correspond to the test papers 311 included in the curved test strip 310 and emits light onto the test papers 311. Thus, it is possible to provide sufficient illuminance to the test papers 311.

Further, the light receiving unit 420 may receive light reflected from the test papers 311. For example, the light receiving unit 420 may be configured as a light receiving sensor with a 16-bit resolution.

The light receiving unit 420 may be an image sensor configured to detect the color of the test paper 311 of the curved test strip 310 based on the light reflected from the test paper 311. For example, the light receiving unit 420 may be an RGB color sensor capable of detecting red, green and blue light, or an RGBW color sensor, such as VEML6040, capable of detecting red, green, blue and white light, using an I²C interface.

Herein, the VEML6040 may be a component having dimensions of 2.0 mm×1.25 mm×1.0 mm (L×W×H), with each channel (R, G, B, W) having a 16-bit resolution.

Furthermore, the light receiving unit 420 and the MCU may be connected by an I²C bus, which allows the mater MCU to read RGBW data detected by the slave light receiving unit 420.

The light emitting unit 410 and the light receiving unit 420 may be appropriately arranged or separated to suppress the direct introduction of light into the light receiving unit 420. Therefore, each light receiving unit 420 may be configured to receive only the reflected light of the light emitted from the corresponding light emitting unit 410.

Meanwhile, the urine analysis device 10 of the present embodiment may further include a substrate unit that supports the sensor units 400. That is, the substrate unit may be provided on an inner side surface of the outer case 100, and the plurality of sensor units 400 may be arranged on the substrate unit at predetermined intervals.

In the present embodiment, the substrate unit may be configured as an FPCB to be arranged on the inner wall of the outer case 100 with the same or similar curvature as the test strip, which is bent along the circumference of the container 300. The substrate unit may be configured to read various types of samples and formed with sufficient flexibility and thinness to maintain the pliability of the material.

After the container 300 is mounted in the inner case 200, the analysis unit (not shown) according to an embodiment of the present disclosure may identify the type of container 300 (11-point kit, 7-point kit, or 4-point kit) based on the number of test items.

The correction unit (not shown) may select available sensor units from among the plurality of sensor units 400 based on data received from the plurality of sensor units 400.

In an embodiment, data related to optical signals received by the plurality of sensor units 400 (e.g., the light receiving units 420) may be transmitted to the correction unit (not shown). The correction unit (not shown) may select available sensor units 400 positioned to best detect the test paper 311 of the test strip from among the plurality of densely arranged sensor units 400.

Table 2 below shows matching data including available sensor units with color sensors for each test paper 311 when the container 300 (11-point kit) includes 11 test papers 311 in the test strip.

TABLE 2

| | Test | Test | |
|---|---|---|---|
| No. | Item | Paper | Available Sensor Unit |
| 1 | UT1 | TP1 | Light Emitting Unit 1, Light Receiving Unit 1 |
| 2 | UT2 | TP2 | Light Emitting Unit 2, Light Receiving Unit 2 |
| 3 | UT3 | TP3 | Light Emitting Unit 3, Light Receiving Unit 3 |
| 4 | UT4 | TP4 | Light Emitting Unit 4, Light Receiving Unit 4 |
| 5 | UT5 | TP5 | Light Emitting Unit 5, Light Receiving Unit 5 |
| 6 | UT6 | TP6 | Light Emitting Unit 6, Light Receiving Unit 6 |
| 7 | UT7 | TP7 | Light Emitting Unit 7, Light Receiving Unit 7 |
| 8 | UT8 | TP8 | Light Emitting Unit 8, Light Receiving Unit 8 |
| 9 | UT9 | TP9 | Light Emitting Unit 9, Light Receiving Unit 9 |
| 10 | UT10 | TP10 | Light Emitting Unit 10, Light Receiving Unit |
| | | | 10 |
| 11 | UT11 | TP11 | Light Emitting Unit 11, Light Receiving Unit |
| | | | 11 |

In the present embodiment, the analysis unit (not shown) analyzes urine based on data received from the available sensor units. Specifically, the urine can be analyzed based on color information received from the sensor units 400, and as described above, the test time can be set according to each test item. When the inner case 200 and the container 300 are mounted in the outer case 100, the analysis unit (not shown) sequentially detects the colors of the test papers 311 sensed by the available sensor units based on the data of the available sensor units. Then, the analysis unit (not shown) compares the detected colors with reference colors for the corresponding test items in the standard colorimetric table, determines the levels of the test items, and derives the test result by analyzing the test items and information of the examinee.

Hereinafter, the test strip including less than 11 test papers 311 will be described.

In the urine analysis device 10 according to an embodiment of the present disclosure, the number of sensor units 400 may be set greater than the number of test papers 311 of the test strip.

Specifically, the sensor units 400 may be arranged at predetermined intervals along one direction of the substrate unit, for example, a circumferential direction of the outer case 100 or the container 300. For example, the number of test papers 311 in the test strip used in the urine analysis device 10 of the present embodiment may be 11, 7, or 4, and the number of sensor units 400 may be 22.

Moreover, the plurality of sensor units 400 may be densely arranged such that a gap between the sensor units 400 is smaller than a width of each individual sensor unit 400 in one direction. Further, the density of the sensor units 400 arranged in the space may be set higher than that of the test papers 311.

Since the sensor units 400, which are greater in number than the test papers 311, are densely arranged as described above, it is possible to analyze various containers 300 with different positions or numbers of test papers 311. Therefore, without the need for separate devices for different containers 300 varying in the types or numbers of test items and the sizes of containers 300, the urine analysis device 10 according to the present embodiment can effectively perform analysis.

In an embodiment of the present disclosure, the substrate unit may be formed to allow deformation. The substrate unit, which may be formed of a ductile material, may include notches between the sensor units 400 to allow stretching or deformation in one direction. As the substrate unit is stretched or deformed, a gap between the plurality of sensor units 400 may vary.

To enable stretching of the substrate unit, the urine analysis device 10 of the present embodiment may further include a connection unit. For example, both ends of the connection unit may be fixed respectively to the outer case 100 and the substrate unit. Also, the connection unit may be elastically deformed in length by user manipulation or applied force and thus can pull or push the substrate unit in one direction.

For example, when the substrate unit is pulled, it may elongate within an allowable range, which increases the gap between the plurality of sensor units 400. When the pulling force is released or a pushing force is applied toward the substrate unit, the gap between the plurality of sensor units 400 may be decreased. Further, by pulling or pushing the substrate unit, the substrate can be brought into close contact with the container 300 such that the distance between the sensor units 400 and the test papers 311 is suitable for analysis.

In the present embodiment, by arranging the plurality of sensor units 400 at a high density and varying the gap between the sensor units 400 in the urine analysis device 10, it is possible to analyze various the containers 300 with different sizes or radius of curvature, and different numbers or positions of test papers 311. Furthermore, the analysis can still be performed even if there is a variation in the mounting position of the container 300 in the inner case 200.

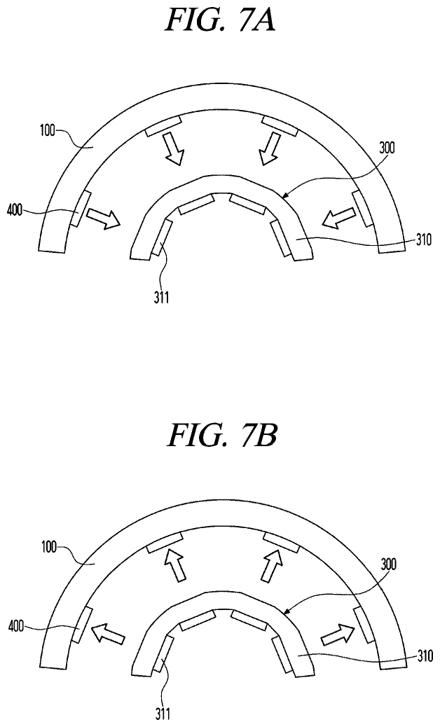

FIG. 7A and FIG. 7B are diagrams for explaining a method of acquiring color information of each of the plurality of test papers 311 by emitting light from the urine analysis device 10 according to an embodiment of the present disclosure.

In FIG. 7A and FIG. 7B, the sensor unit 400 is illustrated as having a configuration in which the light emitting unit 410 and the light receiving unit 420 are integrally formed, but is not limited thereto. The light emitting sensor and the light receiving sensor may also be separately formed.

As shown in the drawings, some of the plurality of sensor units 400, which is densely arranged relative to the test papers 311, may be positioned to correspond to at least one of the test papers 311 included in the curved test strip 310.

As shown in FIG. 7A, the light emitting unit 410 may emit light toward the corresponding test strip. Further, as shown in FIG. 7B, the light receiving unit 420 may receive light reflected from the corresponding test paper 311.

In the present embodiment, even if all the light emitting units 410 emit light toward the test strip, the light receiving unit 420 located at a position corresponding to the test paper 311 receives light effective for analysis. Thus, the sensor unit 400 with the light receiving unit 420 positioned to correspond to the test paper 311 can serve as an available sensor unit. FIG. 7A and FIG. 7B illustrate light emission and reception in the available sensor unit. However, before the available sensor unit is selected, all the light emitting units 410 and light receiving units 420 may operate simultaneously.

Table 3 below shows matching data including available sensor units with color sensors for each test paper 311 when the container 300 (7-point kit) includes 7 test papers 311 in the test strip.

TABLE 3

| | | Color | Test | Test |
|---|---|---|---|---|
| Sensor Unit | Available/Unavailable | Value | Pad | Item |
| Light Emitting Unit 1, | Available | a | TP1 | UT1 |
| Light Receiving Unit 1 | | | | |
| Light Emitting Unit 2, | Unavailable | — | — | — |
| Light Receiving Unit 2 | | | | |
| Light Emitting Unit 3, | Available | b | TP2 | UT2 |
| Light Receiving Unit 33 | | | | |
| Light Emitting Unit 4, | Unavailable | — | — | — |
| Light Receiving Unit 4 | | | | |
| Light Emitting Unit 5, | Available | c | TP3 | UT3 |
| Light Receiving Unit 5 | | | | |
| Light Emitting Unit 6, | Available | d | TP4 | UT4 |
| Light Receiving Unit 6 | | | | |
| Light Emitting Unit 7, | Available | e | TP5 | UT5 |
| Light Receiving Unit 7 | | | | |
| Light Emitting Unit 8, | Unavailable | — | — | — |
| Light Receiving Unit 8 | | | | |
| Light Emitting Unit 9, | Available | f | TP6 | UT6 |
| Light Receiving Unit 9 | | | | |
| Light Emitting Unit 10, | Unavailable | — | — | — |
| Light Receiving Unit 10 | | | | |
| Light Emitting Unit | Available | g | TP7 | UT7 |
| Light Receiving Unit 11 | | | | |

Table 4 below shows matching data including available sensor units with color sensors for each test paper 311 when the container 300 (4-point kit) includes 4 test papers 311 in the test strip.

TABLE 4

| | | Color | Test | Test |
|---|---|---|---|---|
| Sensor Unit | Available/Unavailable | Value | Pad | Item |
| Light Emitting Unit 1, | Unavailable | — | — | — |
| Light Receiving Unit 1 | | | | |
| Light Emitting Unit 2, | Available | a | TP1 | UT1 |
| Light Receiving Unit 2 | | | | |
| Light Emitting Unit 3, | Unavailable | — | — | — |
| Light Receiving Unit 33 | | | | |
| Light Emitting Unit 4, | Unavailable | — | — | — |
| Light Receiving Unit 4 | | | | |
| Light Emitting Unit 5, | Available | b | TP2 | UT2 |
| Light Receiving Unit 5 | | | | |
| Light Emitting Unit 6, | Unavailable | — | — | — |
| Light Receiving Unit 6 | | | | |
| Light Emitting Unit 7, | Unavailable | — | — | — |
| Light Receiving Unit 7 | | | | |
| Light Emitting Unit 8, | Available | c | TP3 | UT3 |
| Light Receiving Unit 8 | | | | |
| Light Emitting Unit 9, | Unavailable | — | — | — |
| Light Receiving Unit 9 | | | | |
| Light Emitting Unit 10, | Available | — | — | — |
| Light Receiving Unit 10 | | | | |
| Light Emitting Unit 11, | Available | d | TP4 | UT4 |
| Light Receiving Unit 11 | | | | |

According to an embodiment of the present disclosure, even if the container 300 varies in type (11-point kit, 7-point kit, or 4-point kit), urine analysis can be performed just by mounting the container 300 in the urine analysis device 10 without the need for a separate matching process between the color sensors and the test papers 311. Therefore, the urine analysis device 10 of the present disclosure provides excellent compatibility since it is possible to relatively freely design the number and size of test papers 311 of the curved test strip 310 attached to the interior of the container 300.

Meanwhile, in the urine analysis device 10 according to an embodiment of the present disclosure, a color value of a sample to be analyzed may be affected and changed due to changes in temperature or humidity of the sensor units 400 and its surroundings. The urine analysis device 10 according to an embodiment of the present disclosure can enhance the accuracy of analysis by measuring temperature, humidity, and inclination of urine through the measurement unit (not shown) and either maintaining the measured values within a predetermined range or compensating for any variations.

Specifically, the measurement unit (not shown) may measure the temperature of the outer case 100, the inner case 200, the sensor unit 400, or its surroundings via a temperature sensor (not shown). Further, the correction unit (not shown) may correct a duty ratio of light emitted by the sensor units 400 based on the temperature measured by the measurement unit (not shown).

That is, when the light emitting unit 410 continuously emits light and increases in temperature, the correction unit (not shown) may increase a duty ratio of the light emitting unit 410 via PWM control. For example, by increasing the duty ratio by 2% for every 2° C. increase in temperature, the output of the light emitting unit 410 can be increased at higher temperatures and deviations in optical data caused by temperature changes can be compensated.

The measurement unit (not shown) may detect whether external light enters the accommodating space of the outer case 100 while the inner case 200 and the container 300 are mounted, using an illuminance sensor (not shown) provided within the accommodating space. When the external light is detected entering the accommodating space, an alarm can be generated via a speaker (not shown), display, or the like.

Also, the measurement unit (not shown) may measure the humidity of the outer case 100, the inner case 200, the sensor unit 400, the substrate unit, or its surroundings. The correction unit (not shown) may compare the measured humidity measured by the measurement unit (not shown) with a predetermined value or range. When the humidity is not equal to a desirable value or within a desirable range, the urine analysis device 10 can generate an alarm, which prompts the user to take corrective action, such as ventilation, to bring the humidity within the desirable range.

Further, the measurement unit (not shown) may measure an inclination of urine collected in the container 300 through a level detection sensor (not shown) provided inside the outer case 100, and may generate an alarm when a measured inclination value exceeds a predetermined inclination value. Then, the user may adjust the position of the urine analysis device 10 to level the container 300.

Furthermore, the correction unit (not shown) of the urine analysis device 10 according to the present embodiment may calculate the distance between the sensor units 400 and the test papers 311. In the urine analysis device 10 of the present embodiment, the container 300 may be inaccurately placed in the inner case 200 or the inner case 200 may be inaccurately placed in the outer case 100 depending on user manipulation. In response, the correction unit (not shown) may calculate the distance between the sensor units 400 and the test papers 311 based on the data received from the sensor units 400. The correction unit (not shown) may compare the calculated distance with a predetermined value or range. When the distance is not equal to a desirable value or within a desirable range, the urine analysis device 10 of the present embodiment can generate an alarm, which prompts the user to take corrective action. For example, the user may be guided to place the container 300 in the inner case 200 or to place the inner case 200 in the outer case 100 again.

Meanwhile, the urine analysis device 10 according to an embodiment of the present disclosure may further include an output unit. For example, the urine analysis device 10 may be equipped with a display to present the analysis result of the tested urine. The output unit can also output the above-described alarm to the user via visual or auditory information. Further, the output unit can transmit the urine analysis result and other data to a device through a communication means.

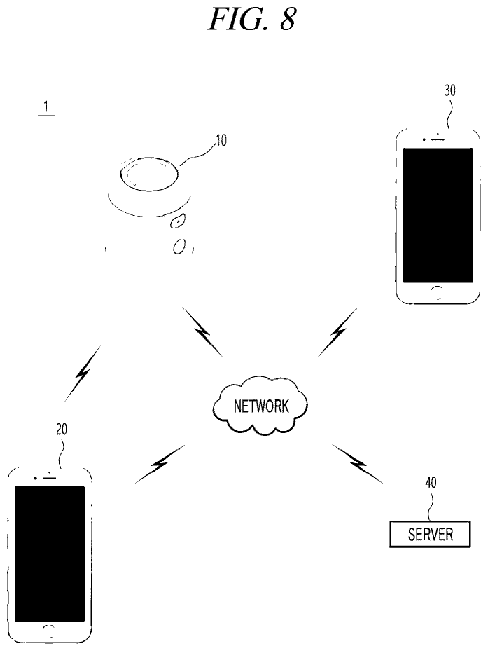

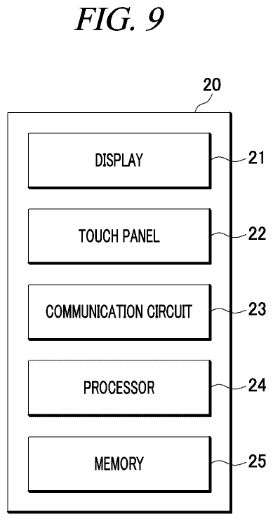

FIG. 8 is a configuration view of a system 1 for providing a health care service related to urine analysis according to an embodiment of the present disclosure. FIG. 9 is a configuration view of an electronic device 20 for providing a health care service related to urine analysis according to an embodiment of the present disclosure.

Referring to FIG. 8 and FIG. 9, the system 1 for providing a health care service related to urine analysis according to an embodiment may include the urine analysis device 10, the electronic device 20, a second electronic device 30, and/or a server 40.

In an embodiment, the electronic device 20 may be capable of directly or indirectly transmitting and receiving signals to and from the urine analysis device 10 via a communication circuit 23. In an embodiment, the electronic device 20 may directly transmit or receive signals to or from the urine analysis device 10. In another embodiment, the electronic device 20 and the urine analysis device 10 may indirectly transmit and receive signals via the server 40 connected through a network.

The urine analysis device 10 according to an embodiment may also include a configuration corresponding to the communication circuit 23 of the electronic device 20. The electronic device 20 according to an embodiment may directly communicate with the urine analysis device 10 or another electronic device 30 through a short-range wireless communication network in a network environment, or may indirectly communicate with the urine analysis device 10, another electronic device 30, or the server 40 through a long-range wireless communication network.

In an embodiment, the signals may include commands or data, and may be transmitted or received directly or indirectly via the server 40 connected through the network between a plurality of electronic devices 20 and the urine analysis device 10.

The second electronic device 30 according to an embodiment may be identical to or different from the electronic device 20. All or some of the operations performed in the electronic device 20 may be performed in the second electronic device 30. In an embodiment, the electronic device 20 may be logged in with a first account, and the second electronic device 30 may be logged in with a second account.

In an embodiment, the electronic device 20 and/or the urine analysis device 10 may communicate with each other via the server 40 connected through the network.

The electronic device 20 according to an embodiment may include a display 21, a touch panel 22, the communication circuit 23, a processor 24, and/or a memory 25.

In an embodiment, the display 21 is a display unit configured to visually provide information to the outside of the electronic device 20, and may include a control circuit for controlling the display unit.

In an embodiment, the touch panel 22 may be an input device configured to receive input from the user corresponding to a display screen on the display 21. The touch panel 22 may include touch circuitry configured to detect a touch, and/or sensor circuitry (e.g., a pressure sensor) configured to measure a magnitude of a force generated by the touch.

In an embodiment, the communication circuit 23 may support establishment of a direct (wired) communication channel and/or a wireless communication channel between the electronic device 20 and the urine analysis device 10, the second electronic device 30, or the server 40, and communication through the established communication channel. The communication circuit 23 may operate independently of the processor 24 (e.g., an application processor), and may include one or more communication processors supporting direct communication and/or wireless communication.

In an embodiment, the communication circuit 23 may include a wireless communication module (e.g., a cellular communication module, a short-range wireless communication module, or a GNSS (Global Navigation Satellite System) communication module) and/or a wired communication module (e.g., a LAN (Local Area Network) communication module or a power line communication module). For example, the communication circuit 23 may communicate with another device via a short-range communication network (e.g., Bluetooth, Wi-Fi Direct, or IrDA (Infrared Data Association)) or a long-range communication network (e.g., a cellular network, the Internet, or a computer network such as LAN or WAN). These various communication circuits 23 may be integrated into a single component (e.g., a single chip) or implemented as a plurality of separate components (e.g., a plurality of chips).

In an embodiment, the processor 24 may execute software (e.g., a program) to control one or more other components (hardware or software components) of the electronic device 20 connected to the processor 24, and to perform various data processing or operations. As a part of the data processing or operations, the processor 24 may load commands and/or data received from other components into the memory 25, process the commands and/or data stored in the memory 25, and store resulting data in the memory 25.

In an embodiment, the processor 24 may include a main processor (e.g., a central processing unit or application processor) and a sub-processor (e.g., a graphics processing unit, image signal processor, sensor hub processor, or communication processor) that can operate independently of or together with the main processor. The sub-processor may consume less power than the main processor and perform specialized functions.

In an embodiment, the memory 25 may store various data required by components (e.g., the processor 24) of the electronic device 20. The data may include, for example, software (e.g., a program), and input and/or output data for any related commands. The memory 25 may include a volatile memory and/or a non-volatile memory. The non-volatile memory may include the internal memory 25 fixedly mounted in the electronic device 20 and a removable external memory. The program may be stored in the memory 25 as software, and may include an operating system, middleware, and/or applications.

The server 40 according to an embodiment may acquire urine analysis result data corresponding to the first account from the electronic device 20 or the urine analysis device 10.

The server 40 according to an embodiment may acquire shared data corresponding to the second account from the electronic device 20. In an embodiment, the shared data may be set for each account with a range of urine analysis result data that can be shared. For example, the range of data to be shared may include whether urine analysis has been performed, the number of analyses performed, time data (e.g., a date), and/or the urine analysis result data (e.g., all or part of a plurality of items).

The server 40 according to an embodiment may extract at least a part of the acquired result data based on the acquired shared data, and may transmit at least a part of the extracted data to the second electronic device 30.

In an embodiment, the server 40 may determine whether different accounts have a family relationship and, when a family relationship is confirmed, may set shared data based on whether predetermined data can be shared and on a range of data that can be shared for the corresponding account.

For example, a user A (father) and a user B (mother) may previously set whether urine analysis result data is to be shared and a range of data that can be shared with a user C (son). The server 40 may set shared data based on whether predetermined data can be shared and the range of data that can be shared, and may provide and present the set shared data in the account of the user C (son). For example, the shared data may include all urine analysis data or data related to the number of urine analyses performed.

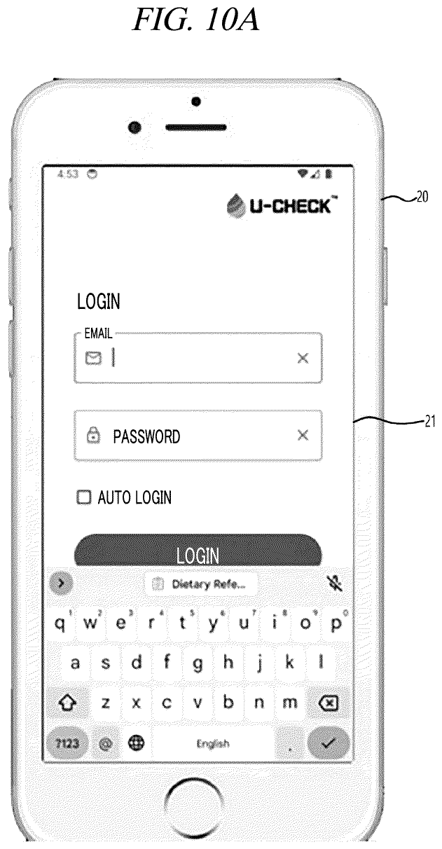

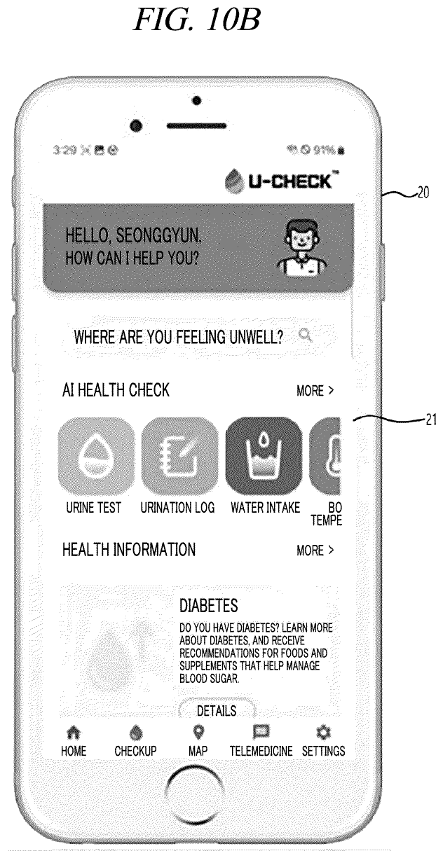

FIG. 10A shows a login screen displayed on the electronic device 20 according to an embodiment of the present disclosure. FIG. 10B shows a main screen displayed on the electronic device 20 according to an embodiment of the present disclosure.

Referring to FIG. 10A and FIG. 10B, the electronic device 20 according to an embodiment may present a screen on the display 21 based on execution of an application or access to a website.

The electronic device 20 according to an embodiment may execute an application or access a website in response to the user's input. In an embodiment, the electronic device 20 may present a login screen of the application or website on the display 21, and may receive, through the touch panel 22, a user ID or email and password corresponding to a user account. In an embodiment, the electronic device 20 may provide personal information security and integrated data management through the user account, and may secure big data for providing various artificial intelligence (AI) services for each account.

The electronic device 20 according to an embodiment may present a main screen on the display 21 in response to a login to the user account. In an embodiment, the main screen may include AI health check and/or health information including icons for urine test, urination log, and water intake.

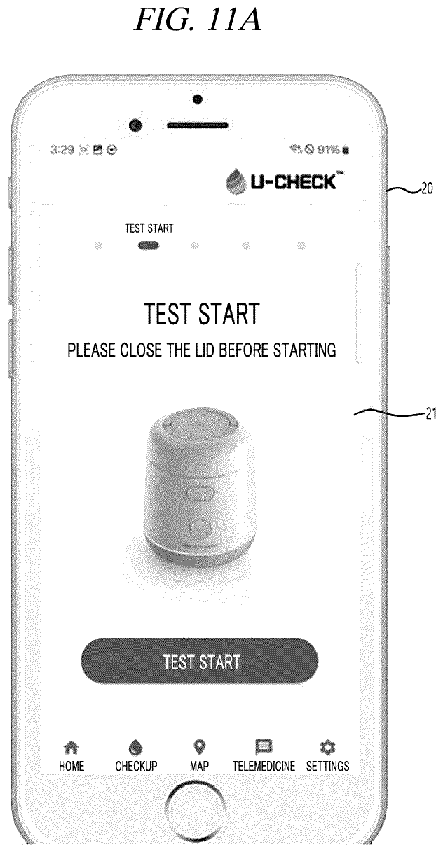

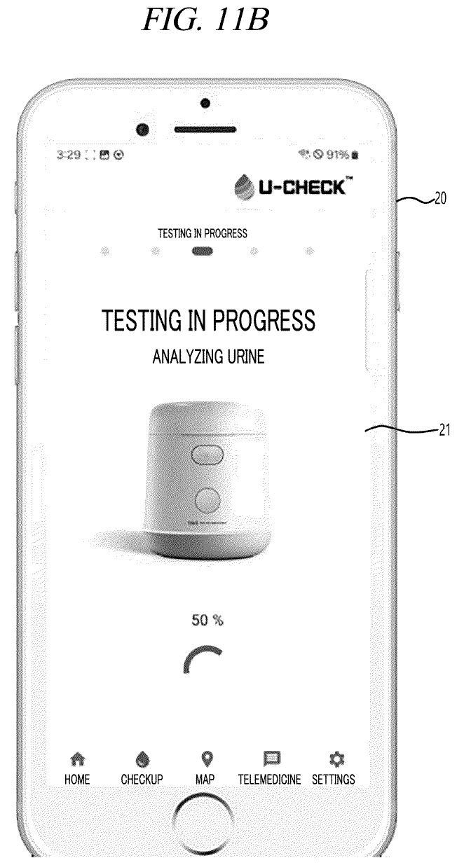

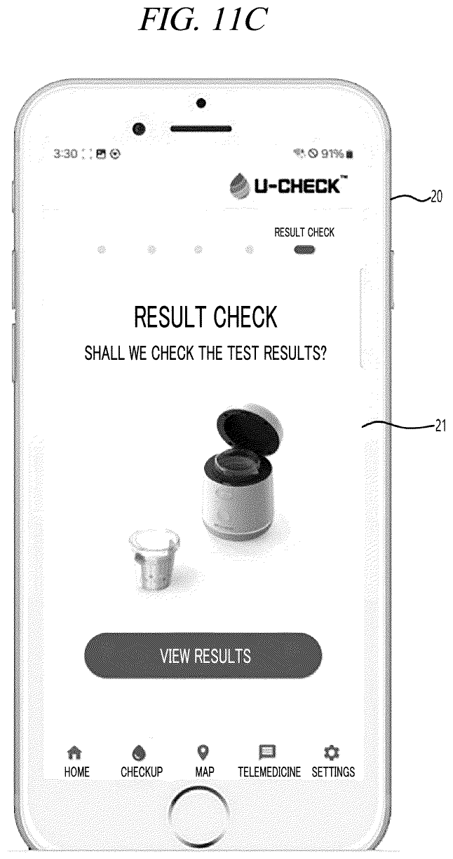

FIG. 11A shows a test start screen displayed on the electronic device 20 according to an embodiment of the present disclosure. FIG. 11B shows a testing in progress screen displayed on the electronic device 20 according to an embodiment of the present disclosure. FIG. 11C shows a result confirmation screen displayed on the electronic device 20 according to an embodiment of the present disclosure.

Referring to FIG. 11A to FIG. 11C, the electronic device 20 according to an embodiment may receive input related to urine analysis from the user, and may transmit and receive signals to and from the urine analysis device 10 based on the acquired input related to urine analysis. In an embodiment, the electronic device 20 may present, on the display 21, screens corresponding to test preparation, test start, testing in progress, result saving, and/or result checking based on the input related to urine analysis.

As shown in the test start screen of FIG. 11A, the electronic device 20 according to an embodiment may receive, through the touch panel 22, a start input for urine analysis from the user. In an embodiment, the electronic device 20 may transmit, through the communication circuit 23, a start signal for the urine analysis to the urine analysis device 10 based on the start input. In an embodiment, the urine analysis device 10 may start a urine analysis based on the received start signal for the urine analysis.

The electronic device 20 according to an embodiment may present, on the display 21, a screen related to a progress status of the urine analysis based on a signal related to the progress status. In an embodiment, the urine analysis device 10 may periodically or in real time transmit the signal related to the progress status of the urine analysis to the electronic device 20, and the electronic device 20 may receive, through the communication circuit 23, the signal related to the progress status of the urine analysis from the urine analysis device 10.

As shown in the testing in progress screen of FIG. 11B, the electronic device 20 according to an embodiment may present, on the display 21, a screen related to the progress status of the urine analysis (e.g., the testing in progress screen). For example, the screen related to the progress status of the urine analysis may display progress status information (e.g., percentage). In an embodiment, when the urine analysis is completed, the electronic device 20 may present a test complete screen on the display 21.

As shown in the test complete screen of FIG. 11C, the electronic device 20 according to an embodiment may receive an input to view the urine analysis result from the user through the touch panel 22.

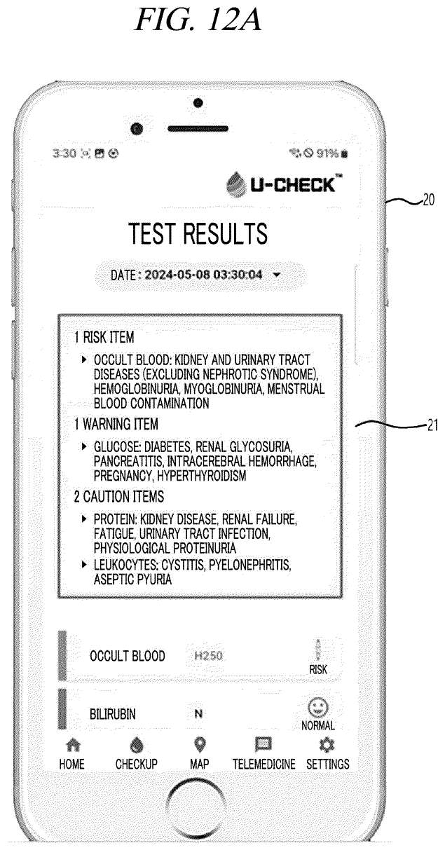

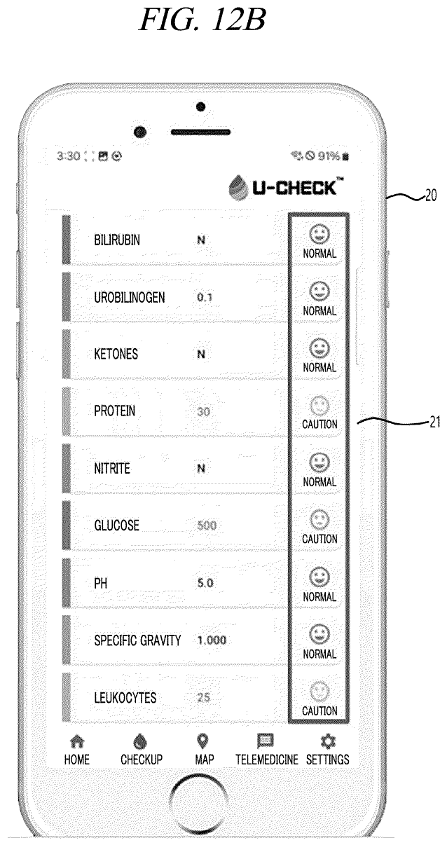

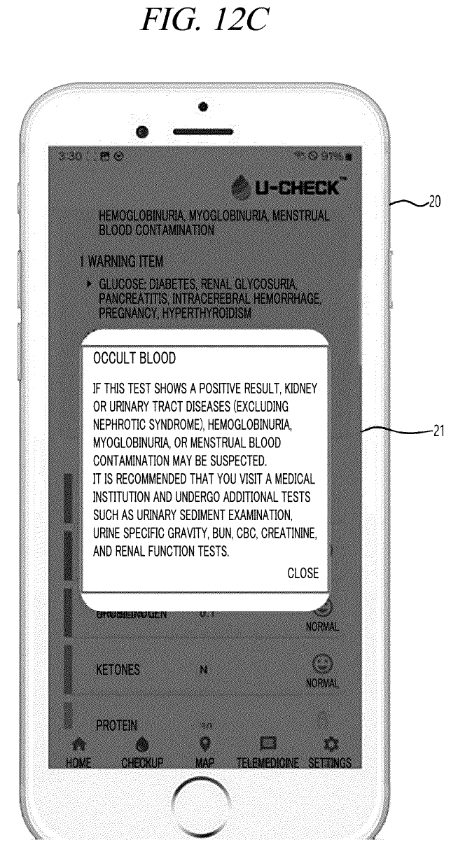

FIG. 12A to FIG. 12C are screens showing urine analysis results displayed on the electronic device 20 according to an embodiment of the present disclosure.

Referring to FIG. 12A to FIG. 12C, when the urine analysis is completed, the urine analysis device 10 according to an embodiment may transmit a signal related to a urine analysis result to the electronic device 20, and the electronic device 20 may receive, through the communication circuit 23, the signal related to the urine analysis result from the urine analysis device 10.

The electronic device 20 according to an embodiment may present, on the display 21, a urine analysis result screen based on the signal related to the result. In an embodiment, when the electronic device 20 acquires an input to view the urine analysis result from the user, the electronic device 20 may display the urine analysis result screen.

In an embodiment, as shown in FIG. 12A, the result screen may include a result in which at least some of a plurality of items corresponding to the signal related to the result are categorized based on severity levels. In an embodiment, the result screen may include a result in which risk items (e.g., occult blood), warning items (e.g., glucose), and caution items (e.g., protein and leukocytes) among a plurality of items corresponding to the signal related to the result are categorized based on severity levels.

In an embodiment, the result screen may include guidance on suspected diseases that are predicted based on at least some of the plurality of items. In an embodiment, the result screen may include guidance on suspected diseases that are predicted according to risk items (e.g., occult blood), warning items (e.g., glucose), and caution items (e.g., protein and leukocytes), such as kidney or urinary tract disease (excluding nephrotic syndrome), hemoglobinuria, myoglobinuria, menstrual blood contamination, diabetes, renal glycosuria, pancreatitis, intracerebral hemorrhage, pregnancy, hyperthyroidism, kidney disease, renal failure, fatigue, urinary tract infection, physiological proteinuria, cystitis, pyelonephritis, and aseptic pyuria.

In an embodiment, as shown in FIG. 12B, the result screen may include results for all or some of the plurality of items corresponding to the signal related to the result. In an embodiment, the result screen may include measurement values and normality determination results (e.g., normal, warning, or caution) for each of the plurality of items.

The electronic device 20 according to an embodiment may receive, through the touch panel 22, a touch input for at least some of the plurality of items while the urine analysis result screen is presented on the display 21.

In an embodiment, the electronic device 20 may present, on the display 21, a screen recommending a customized additional test corresponding to an item based on the touch input for at least some of the plurality of items. In an embodiment, as shown in FIG. 12C, the electronic device 20 may present, on the display 21, a screen recommending a customized additional test corresponding to a specific item (e.g., occult blood) based on a touch input for the specific item.

The electronic device 20 according to an embodiment may store the result screen together with time data of the urine analysis. In an embodiment, the electronic device 20 may present, on the display 21, any one of the stored result screens corresponding to time data selected based on the user's input.

In an embodiment, the result screens may be stored in association with corresponding test dates, and both the test date and the corresponding test result may be stored together. In an embodiment, based on the user's touch input for any one of the test results stored in association with corresponding test dates, the electronic device 20 may present, on the display 21, a result screen corresponding to the test result associated with the selected test date.

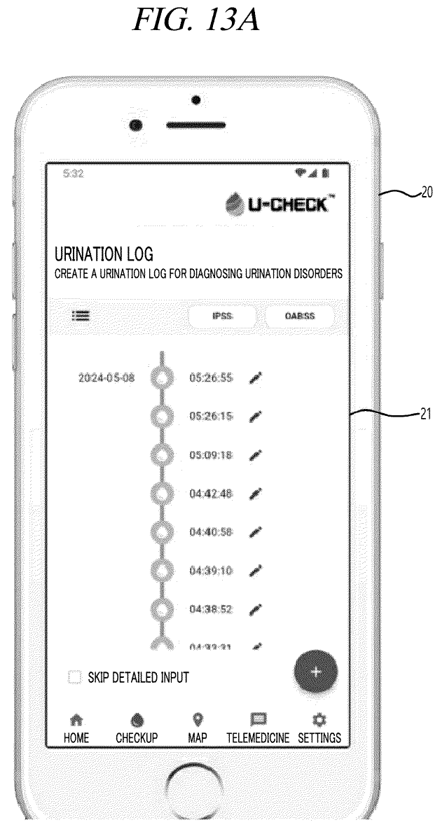

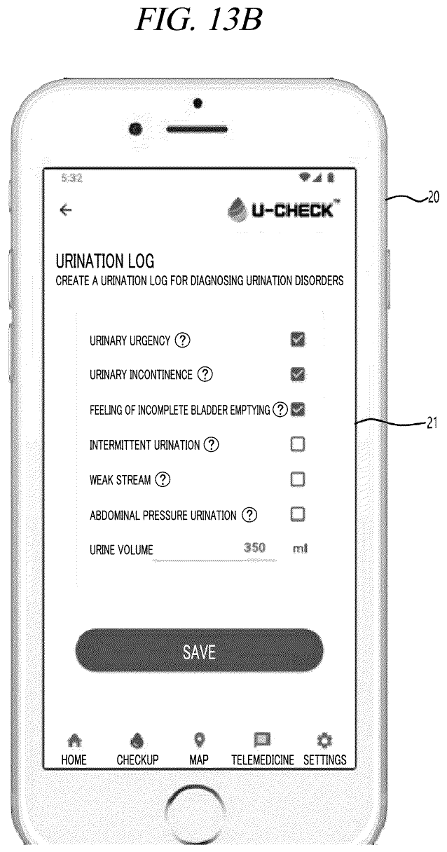

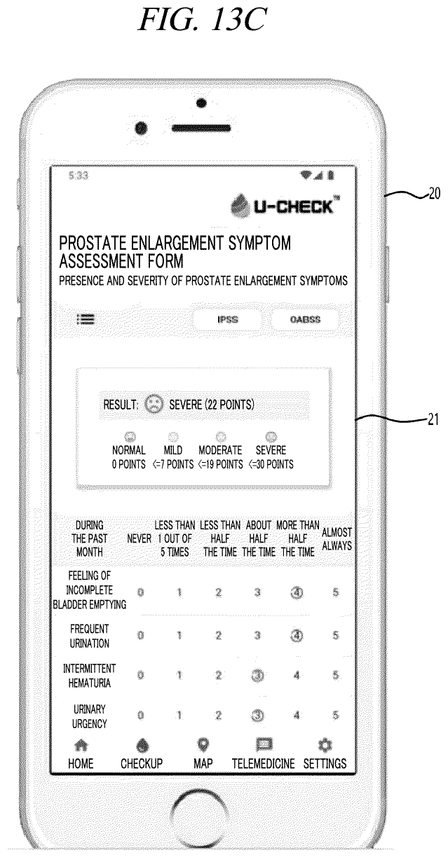

FIG. 13A to FIG. 13C are screens related to a urination log displayed on the electronic device 20 according to an embodiment of the present disclosure.

Referring to FIG. 13A to FIG. 13C, the electronic device 20 according to an embodiment may provide a service related to a urination log based on the user's touch input for a urination log icon.

The electronic device 20 according to an embodiment may acquire urination data from the user through the touch panel 22, and may store the acquired urination data.

In an embodiment, as shown in FIG. 13A, the electronic device 20 may present a urination log screen on the display 21. In an embodiment, the urination log screen may include the urination-related data stored together with time data of urination.

In an embodiment, as shown in FIG. 13B, the electronic device 20 may acquire urination data from the user through the touch panel 22. In an embodiment, the urination data may include a urination volume and information indicating whether predetermined urination characteristics are satisfied. For example, the predetermined urination characteristics may include urinary urgency, urinary incontinence, feeling of incomplete bladder emptying, intermittent urination, weak stream, and/or abdominal pressure urination.

In an embodiment, as shown in FIG. 13C, the electronic device 20 may present, on the display 21, a disease guidance screen including guidance on suspected diseases that are predicted based on the stored urination data.

In an embodiment, the suspected diseases may be analyzed based on a total score calculated according to the urination volume or the number of occurrences of predetermined urination characteristics during a predetermined period. In an embodiment, the suspected diseases may be analyzed based on urination-related data input by the user and stored in the electronic device 20.

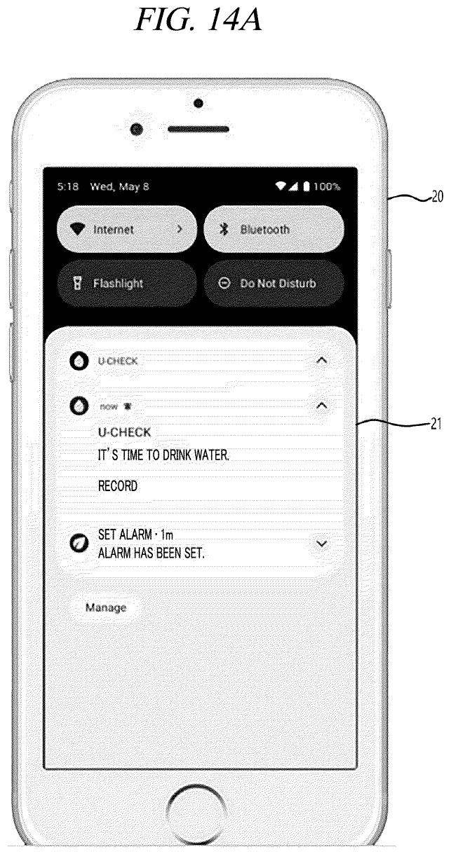

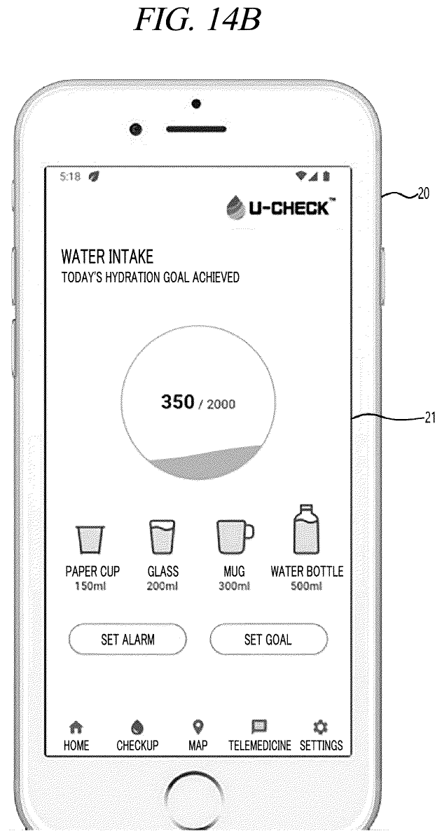

FIG. 14A to FIG. 14C are screens related to water intake displayed on the electronic device 20 according to an embodiment of the present disclosure.

Referring to FIG. 14A to FIG. 14B, the electronic device 20 according to an embodiment may provide a service related to water intake based on the user's touch input for a water intake icon.

The electronic device 20 according to an embodiment may provide, through the display 21, a water intake notification based on a period preset by the user. In an embodiment, the electronic device 20 may provide, through the display 21, a visual notification such as an application alert, badge, or pop-up window.

In an embodiment, the electronic device 20 may receive periodic notification settings including a period preset by the user, and may periodically provide water intake notifications according to the received settings.

In an embodiment, the electronic device 20 may store water intake data based on the user's input corresponding to a notification. In an embodiment, the electronic device 20 may receive, through the touch panel 22, water intake data in response to a water intake notification presented on the display 21, and may store the received data. Accordingly, when the user receives a water intake notification, the user may be reminded to drink water, and may easily input corresponding water intake data in response to the notification.

In an embodiment, the electronic device 20 may present, on the display 21, a target water intake, an actual water intake, and icons of predetermined volumes, and may update the water intake by applying one of the predetermined volumes based on the user's input for the icon.

In an embodiment, as shown in FIG. 14B, the electronic device 20 may present, on the display 21, a target water intake, an actual water intake, and icons representing predetermined volumes. In an embodiment, the target water intake may be set or recommended by the user corresponding to a predetermined period (e.g., one day), and the actual water intake may be stored as an accumulated amount of water intake input by the user during the predetermined period.

In an embodiment, the electronic device 20 may display a plurality of icons representing predetermined volumes (e.g., paper cup, glass, mug, and water bottle). In an embodiment, the electronic device 20 may receive, through the touch panel 22, the user's touch input for any one of the predetermined volume icons, and may update the water intake by applying the predetermined volume based on the user's touch input.

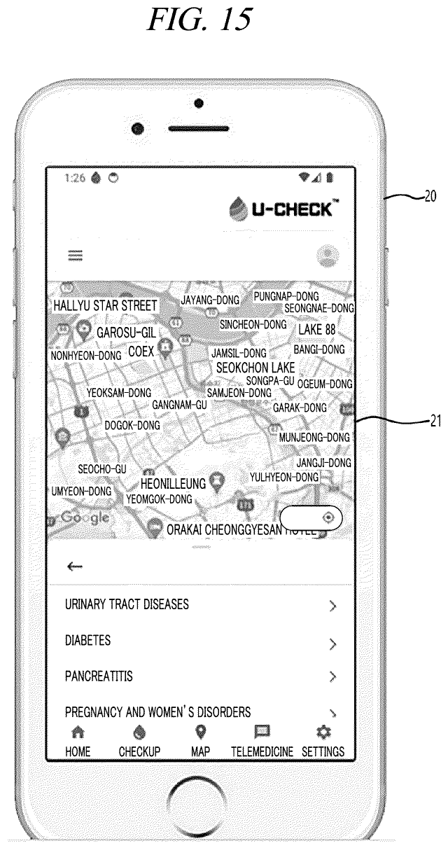

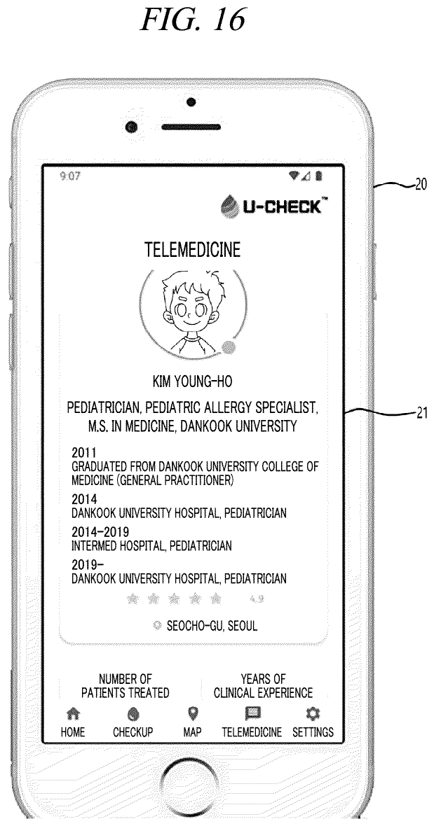

FIG. 15 is a screen for providing medical-institution information displayed on the electronic device 20 according to an embodiment of the present disclosure. FIG. 16 is a screen for providing telemedicine displayed on the electronic device 20 according to an embodiment of the present disclosure.

Referring to FIG. 15 to FIG. 16, the electronic device 20 according to an embodiment may present, on the display 21, guidance on a medical institution based on suspected diseases that are predicted based on at least some of a plurality of items included in a signal related to a result of urine analysis or the user's location.

When the user's touch input for a map is acquired, the electronic device 20 according to an embodiment may present, on the display 21, guidance on a medical institution based on the user's location. In an embodiment, the electronic device 20 may display, on a map or in a list, medical institutions located within a predetermined range from the user's location.

The electronic device 20 according to an embodiment may present, on the display 21, guidance on a medical institution based on suspected diseases. In an embodiment, the suspected diseases may be analyzed based on measurement values and normality determination results (e.g., normal, warning, or caution) for each of a plurality of items included in a signal related to a result of urine analysis.

The electronic device 20 according to an embodiment may present, on the display 21, detailed information (e.g., address, telephone number, operation hours, etc.) of a medical institution based on the user's touch input for the medical institution presented on the display 21.

The electronic device 20 according to an embodiment may present, on the display 21, guidance on medical staff based on a signal related to a result of urine analysis or a result screen. In an embodiment, the result screen may include measurement values and normality determination results (e.g., normal, warning, or caution) for each of the plurality of items included in the signal related to the urine analysis result.

In an embodiment, the electronic device 20 may analyze suspected diseases based on the signal related to the urine analysis result or the result screen, and may present, on the display 21, guidance on medical staff (e.g., a doctor) based on the analyzed suspected diseases.

The electronic device 20 according to an embodiment may transmit, through the communication circuit 23, the signal related to the result or the result screen to the medical staff's device based on the user's input corresponding to the guidance on the medical staff. In an embodiment, the signal related to the urine analysis result or the result screen may be transmitted only to the medical staff selected by the user.

The electronic device 20 according to an embodiment may receive medical data from the medical staff's device through the communication circuit 23, and may present, on the display 21, a telemedicine screen related to the received medical data. In an embodiment, the medical data may be input into the medical staff's device by the medical staff and may be in various forms, such as text, photographs, or drawings.

In an embodiment, the electronic device 20 may present a telemedicine screen on the display 21 based on the user's input. In an embodiment, a default country may be selected on the telemedicine screen based on the location of the electronic device 20, and the electronic device 20 may transmit a request signal to the server 40 according to the default country.

In an embodiment, the server 40 may include a server for each country or may have a database (DB) address for each country. Also, the server 40 may access a country-specific DB corresponding to a country and/or institution requested by the electronic device 20.

In an embodiment, the server 40 may convert medical staff DB and/or medical institution DB of a plurality of countries into a predetermined format and provide them to the electronic device 20. Also, the electronic device 20 may present, on the display 21, the DB converted into the predetermined format.

In an embodiment, after simultaneously accessing the medical staff DBs and the medical institution DBs, the server 40 may read schemas of the medical institution DBs of a plurality of countries and apply schema modification rules corresponding to each country. For example, when a table name is “us_doctors,” the server 40 may convert integer values in a “category_type” column into string values in a “category_name” column of a “doctor” table in the medical staff DB and medical institution DB. In an embodiment, when permission to store data in the medical staff DB and the medical institution DB of a corresponding country is granted, the server 40 may generate or store a country-specific DB therein. In an embodiment, when permission to store data in the medical staff DB and the medical institution DB of a corresponding country is not granted, the server 40 may return the data to the electronic device 20 through a memory (e.g., volatile memory).

In an embodiment, a remote telemedicine service may be a paid service, and the electronic device 20 may receive payment data from the user to perform payment processing.

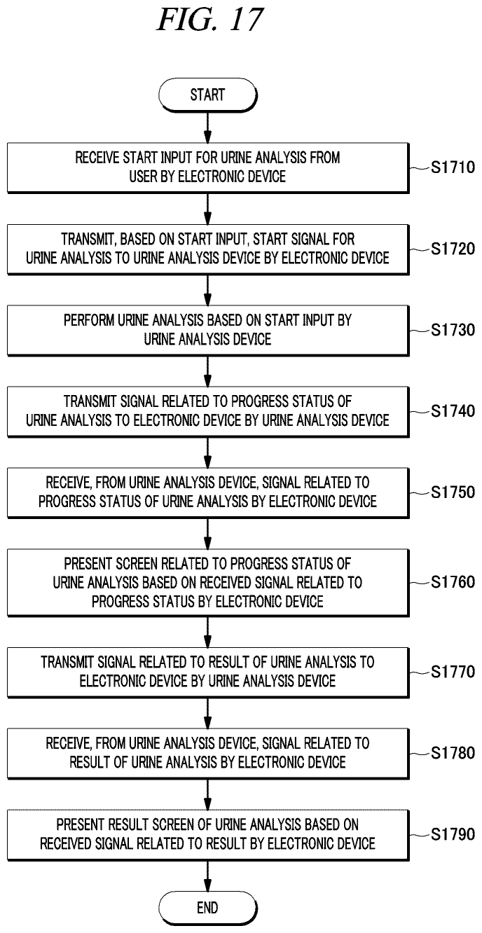

FIG. 17 is a flowchart showing a method for providing a health care service related to urine analysis according to an embodiment of the present disclosure.

Referring to FIG. 17, in an operation 1710, the electronic device 20 according to an embodiment may receive a start input for urine analysis from a user.

In an operation 1720, the electronic device 20 according to an embodiment may transmit, based on the start input, a start signal for the urine analysis to the urine analysis device 10.

In an operation 1730, the urine analysis device 10 according to an embodiment may perform urine analysis based on the start input.

In an operation 1740, the urine analysis device 10 according to an embodiment may transmit a signal related to a progress status of the urine analysis to the electronic device 20.

In an operation 1750, the electronic device 20 according to an embodiment may receive, from the urine analysis device 10, the signal related to the progress status of the urine analysis.

In an operation 1760, the electronic device 20 according to an embodiment may present a screen related to the progress status of the urine analysis based on the received signal related to the progress status.

In an operation 1770, the urine analysis device 10 according to an embodiment may transmit a signal related to a result of the urine analysis to the electronic device 20.

In an operation 1780, the electronic device 20 according to an embodiment may receive, from the urine analysis device 10, the signal related to the result of the urine analysis.

In an operation 1790, the electronic device 20 according to an embodiment may present a result screen of the urine analysis based on the received signal related to the result.

The method for providing a health care service related to urine analysis in the electronic device 20 that provides the above-described health care service related to urine analysis can be implemented as a computer program stored in a computer-readable storage medium to be executed by a computer or a storage medium including instructions executable by a computer. Also, the above-described method can be implemented as a computer program stored in a computer-readable storage medium to be executed by a computer.

A computer-readable medium can be any usable medium which can be accessed by the computer and includes all volatile/non-volatile and removable/non-removable media. Further, the computer-readable medium may include all computer storage media. The computer storage media include all volatile/non-volatile and removable/non-removable media embodied by a certain method or technology for storing information such as computer-readable instruction code, a data structure, a program module or other data.

The functions realized by the components described in the specification herein may be implemented in general-purpose processors, special-purpose processors, integrated circuits, application specific integrated circuits (ASICs), central processing units (CPUs), circuits, and/or processing circuitry including combinations of these programmed to realize the described functions. A processor contains transistors or other circuits and is considered a circuit or processing circuit. The processor may be a programmed processor that executes a program stored in memory.

In the specification herein, a circuit, part, unit, or means is hardware programmed or executing hardware to realize the described function. The hardware may be any hardware disclosed herein or any hardware known to be programmed or executing the described functions.

If the hardware is a processor that is considered a circuit type, then the circuit, the part, means or unit in question is a combination of hardware and software used to configure the hardware and or processor.

The above description of the present disclosure is provided for the purpose of illustration, and it would be understood by a person with ordinary skill in the art that various changes and modifications may be made without changing technical conception and essential features of the present disclosure. Thus, it is clear that the above-described examples are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

### EXPLANATION OF CODES

1: Urine analysis system
10: Urine analysis device
100: Outer case
110: Outer cover
200: Inner case
210: Retaining part
300: Container
310: Curved test strip
311: Test paper
320: Container cover
330: Flow path
400: Sensor unit
410: Light emitting unit
420: Light receiving unit
20: Electronic device
40: Server

We claim:

1. A system for providing a health care service related to urine analysis, comprising:
a urine analysis device that includes a curved test strip which is attached along an inner or outer circumferential surface of a container into which urine is collected and includes a plurality of test papers provided to test the collected urine, a sensor unit which includes a light emitting unit configured to emit light onto the plurality of test papers and a light receiving unit configured to detect a color of each test paper based on the light reflected from each of the plurality of test papers, and an analysis unit which analyzes the urine based on the color information of the test papers detected by the light receiving unit; and
an electronic device that includes a communication circuit configured to directly or indirectly transmit and receive signals with the urine analysis device, a display, and a touch panel integrally coupled with the display,
the electronic device is configured to:
transmit, through the communication circuit, a start signal for the urine analysis to the urine analysis device;
present, on the display, a screen related to a progress status of the urine analysis based on a signal related to the progress status of the urine analysis and received from the urine analysis device; and
present, on the display, a result screen of the urine analysis based on a signal related to a result of the urine analysis and received from the urine analysis device, and
the urine analysis device is configured to:
perform urine analysis based on the color information of the test papers in response to the start signal for the urine analysis;
transmit the signal related to the progress status of the urine analysis to the electronic device; and
transmit the signal related to the result of the urine analysis to the electronic device.

2. The system of claim 1,
wherein the urine analysis device further includes:
an outer case; and
an inner case detachably disposed inside the outer case, and
the container is mounted in the inner case, and
an accommodating space of the outer case has a cylindrical shape concentric with the curved test strip.

3. The system of claim 2,
wherein the sensor unit is formed on a substrate that is formed of a ductile material on an inner wall of the outer case with the same curvature as the curved test strip, which is bent along the circumference of the container.

4. The electronic device of claim 1,
wherein the sensor unit is arranged on at least a part of an inner wall of the outer case and oriented toward the center of the accommodating space of the outer case.

5. The system of claim 1,
the sensor unit is formed on a substrate that is formed of a ductile material on at least a part of an inner wall of a cylindrical accommodating space.

6. The system of claim 1,
wherein the result screen includes a result in which at least some of a plurality of items corresponding to the signal related to the result are categorized based on severity levels.

7. The system of claim 6,
wherein the electronic device is configured to:
receive, through the touch panel, a touch input for the at least some of the plurality of items; and
present, on the display, a screen recommending a customized additional test corresponding to the at least some of the plurality of items based on the touch input.

8. The system of claim 6,
wherein the result screen includes guidance on suspected diseases that are predicted based on the at least some of the a plurality of items.

9. The system of claim 1,
wherein the electronic device is configured to:
acquire urination data from a user through the touch panel;
store the acquired urination data; and
present, on the display, a disease guidance screen including guidance on suspected diseases that are predicted based on the stored urination data.

10. The system of claim 9,
wherein the urination data includes a urination volume and information indicating whether predetermined urination characteristics are satisfied, and
the suspected diseases are analyzed based on a total score calculated according to the urination volume or the number of occurrences of predetermined urination characteristics during a predetermined period.

11. The system of claim 1,
wherein the electronic device is configured to:
provide, on the display, a water intake notification based on a period preset by a user; and
store water intake data based on the user's input corresponding to the notification.

12. The system of claim 11,
wherein the electronic device is configured to:
present, on the display, a target water intake, an actual water intake, and icons of predetermined volumes corresponding to the user; and
update the water intake by applying one of the predetermined volumes based on the user's input for the icon.

13. A method for providing a health care service related to urine analysis, comprising:
transmitting a start signal for the urine analysis to a urine analysis device by an electronic device;
performing urine analysis in response to the start signal by the urine analysis device;
transmitting a signal related to a progress status of the urine analysis to the electronic device by the urine analysis device;
presenting a screen related to the progress status of the urine analysis based on the signal related to the progress status and received from the urine analysis device by the electronic device;
transmitting a signal related to a result of the urine analysis to the electronic device by the urine analysis device; and
presenting a result screen of the urine analysis based on the signal related to the result of the urine analysis and received from the urine analysis device by the electronic device,
wherein the urine analysis device includes a curved test strip which is attached along an inner or outer circumferential surface of a container into which urine is collected and includes a plurality of test papers provided to test the collected urine, a sensor unit which includes a light emitting unit configured to emit light onto the plurality of test papers and a light receiving unit configured to detect a color of each test paper based on the light reflected from each of the plurality of test papers, and an analysis unit which analyzes the urine based on the color information of the test papers detected by the light receiving unit.
